(19) United States
(12) Patent Application Publication
(10) Pub. No.: US 2024/0207252 A1
(43) Pub. Date: Jun. 27, 2024

(54) AEROSOL-FORMING SUBSTRATE AND AEROSOL-GENERATING SYSTEM

(71) Applicant: SHENZHEN FIRST UNION TECHNOLOGY CO., LTD., Shenzhen, Guangdong Province (CN)

(72) Inventors: Jin LU, Shenzhen, Guangdong Province (CN); Zhongli XU, Shenzhen, Guangdong Province (CN); Yonghai LI, Shenzhen, Guangdong Province (CN)

(73) Assignee: SHENZHEN FIRST UNION TECHNOLOGY CO., LTD., Shenzhen, Guangdong Province (CN)

(21) Appl. No.: 18/554,479

(22) PCT Filed: Apr. 12, 2022
(86) PCT No.: PCT/CN2022/086384

§ 371 Date: Oct. 9, 2023

(30) Foreign Application Priority Data
Apr. 12, 2021 (CN) 202110398742.4

(51) Int. Cl. A61K 31/475; A24B 15/167; A24B 15/30; A24B 15/38; A24F 40/10; A61K 9/00; A61K 47/10; A61M 11/04

(52) U.S. Cl. CPC A61K 31/475; A24B 15/167; A24B 15/303; A24B 15/385; A24F 40/10; A61K 9/0073; A61K 47/10; A61M 11/042

(57) ABSTRACT

An aerosol-forming substrate and an aerosol generation system are provided. The aerosol-forming substrate includes yohimbine or a pharmaceutically acceptable salt of yohimbine or yohimbine hydrate. Based on a total mass of the aerosol-forming substrate, a mass percent of the yohimbine or the pharmaceutically acceptable salt of the yohimbine or the yohimbine hydrate is in a range of 0.1%-10%, preferably 0.1%-8%, further preferably 0.1%-6%, further preferably 0.1%-5%, further preferably 0.5%-5%, further preferably 1%-5%, and further preferably 2%-5%. In the aerosol-forming substrate containing yohimbine or a pharmaceutically acceptable salt of yohimbine or yohimbine hydrate provided in this application, no carbonization occurs after repeated heating by using an electronic vaporization device, and a conversion rate of yohimbine is relatively high, so that a content of yohimbine is close to a dosage through oral administration or injection in current clinical trials, thereby guaranteeing safety.

### CROSS-REFERENCE TO RELATED APPLICATIONS

This application claims priority to Chinese Patent Application No. 202110398742.4, filed with the China National Intellectual Property Administration on Apr. 12, 2021 and entitled “AEROSOL-FORMING SUBSTRATE AND AEROSOL GENERATION SYSTEM”, which is incorporated herein by reference in its entirety.

### TECHNICAL FIELD

This application relates to the technical field of vaporization, and in particular, to an aerosol-forming substrate and an aerosol generation system.

### BACKGROUND

Yohimbine is an alkaloid of natural origin. The ingredient was first extracted from the bark of the Rubiaceae Corunant Yohimbine tree in West Africa. The chemical formula for yohimbine is methyl 17alpha-hydroxyyohimban-16alpha-carboxylate, and the molecular formula is C21H26N2O3.

Literature research shows that yohimbine has a wide range of pharmacological effects and has been developed for clinical treatment of diseases such as arteriosclerosis and rheumatism. The most obvious pharmacological effect is to treat male sexual dysfunction.

At present, most clinical trials are administered orally or by injection. Studies have proved that yohimbine is a one-compartment metabolic model. After a single oral administration, the metabolism in vivo is boosted, and Tmax and t1/2 are both less than 1 h. Since the oral bioavailability is very low and the difference is obvious, the average value is from 22.3% to 33%. Therefore, it is necessary to develop inhalation and administration routes to improve bioavailability.

During implementation of this application, the inventor found that: the patent document with the Application Publication No. of CN112155255A discloses that an evaporable material may contain plant medicines and/or nutrients, for example: yohimbine. In some implementations, the evaporable material is at least 50% soluble (by weight) in any suitable carrier solvent. The solvent is, for example, glycol (such as propanediol and vegetable glycerol), ethylene glycol, dipropylene glycol, trimethylene glycol, ethanol, and a combination thereof.

The disclosed solution has the problems that, on the one hand, the solubility of yohimbine in the carrier solvent is poor, and at least 50% (by weight) of yohimbine cannot be completely dissolved; whether the evaporable material containing the weight can be evaporated and the influence on an electronic evaporator device cannot be verified from this document; and even if the evaporable material can be evaporated, the conversion rate thereof is also very low. On the other hand, the dosage of the evaporable material containing at least 50% (by weight) yohimbine (even if the conversion rate is very low) is very large for users, far exceeding the dosage of administration orally or by injection in present clinical trials, and the safety cannot be guaranteed.

### SUMMARY

During implementation of this application, the inventor finds that a first aspect of this application is intended to provide an aerosol-forming substrate with little influence on an electronic vaporization device, a high conversion rate, and guaranteed safety. The aerosol-forming substrate includes yohimbine or a pharmaceutically acceptable salt of yohimbine or yohimbine hydrate.

Based on a total mass of the aerosol-forming substrate, a mass percent of the yohimbine or the pharmaceutically acceptable salt of the yohimbine or the yohimbine hydrate is in a range of 0.1%-10%, preferably 0.1%-8%, further preferably 0.1%-6%, further preferably 0.1%-5%, further preferably 0.5%-5%, further preferably 1%-5%, and further preferably 2%-5%.

In some implementations, the pharmaceutically acceptable salt is selected from at least one of the following:
hydrochloride, hydrobromide, benzoate, hydrofluoride, sulfate, nitrate, phosphate, formate, acetate, propionate, oxalate, malonate, succinate, fumarate, maleate, lactate, malate, tartrate, citrate, picrate, mesylate, esilate, benzene sulfonate, aspartate, and glutamate.

In some implementations, the pharmaceutically acceptable salt includes benzoate.

In some implementations, the aerosol-forming substrate further includes a solvent, where the solvent is selected from at least one of the following:
propanediol, butanediol, pentanediol, hexanediol, 1,2,4-butanetriol, dipropylene glycol, diethylene glycol, triethylene glycol, polyethylene glycol 200, polyethylene glycol 400, glycerol triacetate, glycerol, dipropylene glycol ether, ethanol, water, triethyl citrate, caprylic capric triglyceride, isopropanol, sweet orange oil, lemon oil, peppermint oil, palm oil, peanut oil, corn oil, salad oil, xylitol, sorbitol, and erythritol.

In some implementations, the solvent includes propanediol, and a mass percent of the propanediol in the aerosol-forming substrate is in a range of 20%-60%, preferably 20%-50%, further preferably 20%-45%, further preferably 25%-45%, further preferably 30%-45%, and further preferably 35%-45%.

In some implementations, the solvent includes glycerol, and a mass percent of the glycerol in the aerosol-forming substrate is in a range of 10-60%, preferably 10%-50%, further preferably 10%-40%, further preferably 20%-40%, and further preferably 30%-40%.

In some implementations, a mass ratio of the propanediol to the glycerol is greater than 1, preferably 1.1-4:1, and further preferably 2-4:1.

In some implementations, the aerosol-forming substrate further includes a flavoring agent.

The flavoring agent in the aerosol-forming substrate is added according to the conventional amount required for flavoring, and the mass percent is in a range of 1-20%, preferably 5%-20%, further preferably 10%-20%, and further preferably 15%-20%. The flavoring agent is selected from at least one of the following:
nerol, trans-2-hexenol, linalool, benzyl alcohol, 1-hexanol, leaf alcohol, alpha-terpineol, citronellol, beta-phenylethyl alcohol, linalool oxide, geraniol, isoamyl alcohol, octanol, hexanol, decyl alcohol, cinnamyl alcohol, heptanol, eugenol, maltol, ethyl maltol, thymol, isoeugenol, 2-methylbutyric acid, malic acid, n-valeric acid, hexanoic acid, edible acetic acid, n-caprylic acid, strawberry acid, butyric acid, citric acid, propionic acid, 3-methylvaleric acid, isovaleric acid, isoamyl acetate, amyl formate, geranyl formate, butyl formate, benzyl formate, cis-3-Hexenyl formate, gamma-Decanolactone, delta-Nonalactone, gamma-Octalactone, gamma-Heptalactone, gamma-Unsecalactone, delta-Dodecalactone, benzaldehyde, strawberry aldehyde, cinnamyl aldehyde, furfural, citral, acetaldehyde, 3-(Methylthio)propionaldehydel, 3-Mercapto-2-methylpentanal, isobutyraldehyde, trans-2-Octenal, trans-2-Nonenal, trans-2-Decenal, trans, trans-2,4-heptadienal, 2,5-Dimethyl pyrazine, 2-Acetylfuran, 2-Ethyl-3,(5 or 6)-dimethylpyrazine, 2,3,5,6-Tetramethylpyrazine, 2,3,5-Trimethylpyrazine, 2-acetyl pyrazine, acetophenone, beta-ionone, Damascenone 2 (Firmenich), butanedione, alpha-ionone, acetoin (acetyl methyl carbinol), acetoin (acetyl methyl alcohol), methyl heptenone, vanillin, ethyl vanillin, dihydrocoumarin, raspberry ketone, anisole, cedryl methyl ether, methyl 2-methyl-3-furyl disulfide, wintergreen oil, clove bud oil, 10-fold sweet orange oil, rosewood oil, geranium oil, bitter almond oil, clove basil oil, ethyl vanillin, dihydrocoumarin, raspberry ketone, trans-2-ethoxy-5-(1-propenyl)phenol, tolu concrete, Peru concrete, oak concrete, espresso supercritical extraction extract (water-soluble), cocoa extract, coffee tincture, angelica pubescens tincture, Pandan extract, vanilla extract, Labdanum concrete, orris oil or concrete, jasmine concrete, tree moss concrete (amber fragrance), tamarind extract, Zimbabwe tobacco extract, tobacco essential oil, burley tobacco extract, flue-cured tobacco absolute A, flue-cured tobacco top note spice extract, and sun-dried top note spice extract.

In some implementations, the aerosol-forming substrate does not comprise nicotine and/or nicotine salts.

A second aspect of this application provides an aerosol generation system. The aerosol generation system includes an electronic vaporization device and the aerosol-forming substrate. The electronic vaporization device is configured to heat and vaporize the aerosol-forming substrate to generate an aerosol.

In the aerosol-forming substrate containing yohimbine or a pharmaceutically acceptable salt of yohimbine or yohimbine hydrate provided in this application, no carbonization occurs after repeated heating by using an electronic vaporization device, and a conversion rate of yohimbine is relatively high, so that a content of yohimbine is close to a dosage through oral administration or injection in current clinical trials, thereby guaranteeing safety.

### BRIEF DESCRIPTION OF THE DRAWINGS

FIG. 1 is a schematic diagram of HPLC detection results according to an implementation of this application;

FIG. 2 is a schematic diagram of a vaporizer in an electronic vaporization device before a carbonization experiment according to an implementation of this application; and

FIG. 3 is a schematic diagram of a vaporizer in an electronic vaporization device after a carbonization experiment according to an implementation of this application.

### DETAILED DESCRIPTION

This application is to be further described with reference to the following embodiments.

### Implementation I:

Implementation I of this application provides an aerosol-forming substrate. The aerosol-forming substrate includes yohimbine or a pharmaceutically acceptable salt of yohimbine or yohimbine hydrate.

Based on a total mass of the aerosol-forming substrate, a mass percent of the yohimbine or the pharmaceutically acceptable salt of the yohimbine or the yohimbine hydrate is in a range of 0.1%-10%, preferably 0.1%-8%, further preferably 0.1%-6%, further preferably 0.1%-5%, further preferably 0.5%-5%, further preferably 1%-5%, and further preferably 2%-5%.

In some implementations, the pharmaceutically acceptable salt is selected from at least one of the following:
hydrochloride, hydrobromide, benzoate, hydrofluoride, sulfate, nitrate, phosphate, formate, acetate, propionate, oxalate, malonate, succinate, fumarate, maleate, lactate, malate, tartrate, citrate, picrate, mesylate, esilate, benzene sulfonate, aspartate, and glutamate.

In some implementations, the pharmaceutically acceptable salt includes benzoate.

In some implementations, the aerosol-forming substrate further includes a solvent, where the solvent is selected from at least one of the following:
propanediol, butanediol, pentanediol, hexanediol, 1,2,4-butanetriol, dipropylene glycol, diethylene glycol, triethylene glycol, polyethylene glycol 200, polyethylene glycol 400, glycerol triacetate, glycerol, dipropylene glycol ether, ethanol, water, triethyl citrate, caprylic capric triglyceride, isopropanol, sweet orange oil, lemon oil, peppermint oil, palm oil, peanut oil, corn oil, salad oil, xylitol, sorbitol, and erythritol.

In the implementation, propanediol includes 1,2-propanediol and 1,3-propanediol. Butanediol includes 1,2-butanediol, 1,3-butanediol, 1,4-butanediol, and 2,3-butanediol. Pentanediol includes 1,2-pentanediol, 1,4-pentanediol, 2,4-pentanediol, and 1,5-pentanediol. Hexylene glycol includes 1,2-hexanediol, 1,6-hexanediol, and 2,5-hexanediol.

In some implementations, the solvent includes propanediol, and a mass percent of the propanediol in the aerosol-forming substrate is in a range of 20%-60%, preferably 20%-50%, further preferably 20%-45%, further preferably 25%-45%, further preferably 30%-45%, and further preferably 35%-45%.

In some implementations, the solvent includes glycerol, and a mass percent of the glycerol in the aerosol-forming substrate is in a range of 10-60%, preferably 10%-50%, further preferably 10%-40%, further preferably 20%-40%, and further preferably 30%-40%.

In some implementations, a mass ratio of the propanediol to the glycerol is greater than 1, preferably 1.1-4:1, and further preferably 2-4:1.

In some implementations, the aerosol-forming substrate further includes a flavoring agent, and a mass percent of the flavoring agent in the aerosol-forming substrate is in a range of 1%-20%, preferably 5%-20%, further preferably 10%-20%, and further preferably 15%-20%.

In some implementations, the flavoring agent is selected from at least one of the following:
nerol, trans-2-hexenol, linalool, benzyl alcohol, 1-hexanol, leaf alcohol, alpha-terpineol, citronellol, beta-phenylethyl alcohol, linalool oxide, geraniol, isoamyl alcohol, octanol, hexanol, decyl alcohol, cinnamyl alcohol, heptanol, eugenol, maltol, ethyl maltol, thymol, isoeugenol, 2-methylbutyric acid, malic acid, n-valeric acid, hexanoic acid, edible acetic acid, n-caprylic acid, strawberry acid, butyric acid, citric acid, propionic acid, 3-methylvaleric acid, isovaleric acid, isoamyl acetate, amyl formate, geranyl formate, butyl formate, benzyl formate, cis-3-Hexenyl formate, gamma-Decanolactone, delta-Nonalactone, gamma-Octalactone, gamma-Heptalactone, gamma-Unsecalactone, delta-Dodecalactone, benzaldehyde, strawberry aldehyde, cinnamyl aldehyde, furfural, citral, acetaldehyde, 3-(Methylthio)propionaldehydel, 3-Mercapto-2-methylpentanal, isobutyraldehyde, trans-2-Octenal, trans-2-Nonenal, trans-2-Decenal, trans, trans-2,4-heptadienal, 2,5-Dimethyl pyrazine, 2-Acetylfuran, 2-Ethyl-3,(5 or 6)-dimethylpyrazine, 2,3,5,6-Tetramethylpyrazine, 2,3,5-Trimethylpyrazine, 2-acetyl pyrazine, acetophenone, beta-ionone, Damascenone 2 (Firmenich), butanedione, alpha-ionone, acetoin (acetyl methyl carbinol), acetoin (acetyl methyl alcohol), methyl heptenone, vanillin, ethyl vanillin, dihydrocoumarin, raspberry ketone, anisole, cedryl methyl ether, methyl 2-methyl-3-furyl disulfide, wintergreen oil, clove bud oil, 10-fold sweet orange oil, rosewood oil, geranium oil, bitter almond oil, clove basil oil, ethyl vanillin, dihydrocoumarin, raspberry ketone, trans-2-ethoxy-5-(1-propenyl)phenol, tolu concrete, Peru concrete, oak concrete, espresso supercritical extraction extract (water-soluble), cocoa extract, coffee tincture, angelica pubescens tincture, Pandan extract, vanilla extract, Labdanum concrete, orris oil or concrete, jasmine concrete, tree moss concrete (amber fragrance), tamarind extract, Zimbabwe tobacco extract, tobacco essential oil, burley tobacco extract, flue-cured tobacco absolute A, flue-cured tobacco top note spice extract, and sun-dried top note spice extract.

It should be noted that, the flavoring agent is not limited to substances listed above, and any flavoring agent that conforms to FEMA coding and CAS coding is applicable.

In some implementations, the aerosol-forming substrate does not comprise nicotine and/or nicotine salts.

### I. HPLC Test:

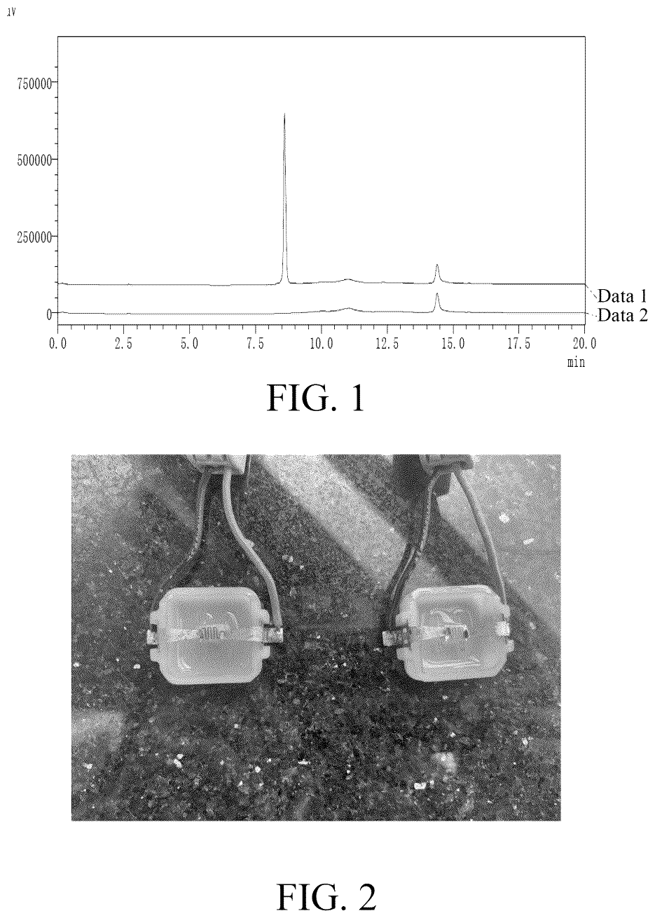

According to the items and parameters shown in the following table, samples with yohimbine and samples without yohimbine are respectively tested through HPLC, and test results may be shown in FIG. 1. Data 1 in the figure is a schematic chromatogram of a sample with yohimbine, and data 2 is a schematic chromatogram of a sample without yohimbine.

| Item | Parameter | |
|---|---|---|
| Chromatographic | InertSustain C18, 4.6 × 250 mm, 3.5 μm | |
| column | | |
| Mobile phase | Acetonitrile-water = 40:60 | |
| Test wavelength | 278 | nm |
| Flow rate | 1.0 | ml/min |
| Column temperature | 40° | C. |

### II. Carbonization Experiment

The aerosol-forming substrate of this embodiment is heated (heated for 3 s every 27 s) by using an electronic vaporization device with a ceramic vaporizer, and a number of heating (or puffs) is 400 times. The aerosol-forming substrate in this embodiment is composed of 1.5% of yohimbine, 40% of propanediol, 37% of glycerol, and 20% of the flavoring agent.

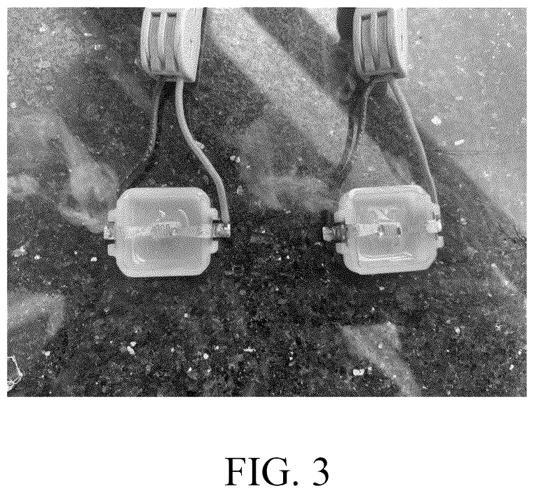

FIG. 2 is a schematic diagram of a vaporizer before the electronic vaporization device heats the aerosol-forming substrate. FIG. 3 is a schematic diagram of a vaporizer after the electronic vaporization device heats the aerosol-forming substrate. Through observation with naked eyes, it can be seen that no gelatinization and carbon deposition occurs in the vaporizer. During the experiment, the experimenter does not notice the odor during inhalation.

During the experiment, an intake of TPM each time is about 5-6 mg. 4% of yohimbine is used as an example. A content of yohimbine each time is about 0.24 mg. It may be learned by looking up relevant information that the commercially available yohimbine tablets (taken orally) are 5.4 mg each time, and assuming its bioavailability is 50%, the intake is about 2.7 mg each time. Therefore, an intake of 10 puffs of a user through the electronic vaporization device is about 2.4 mg (the bioavailability of vaporization intake is much higher than that of oral administration), which is close to the dosage of oral administration in current clinical trials, and the safety can be guaranteed.

### III. Conversion Rate Experiment

### Experimental Test Method:

Embodiment 1: The aerosol-forming substrate is composed of 10 wt % yohimbine, 40 wt % propanediol, and 37 wt % glycerol, and the balance is a flavoring agent.

Embodiment 2: The aerosol-forming substrate is composed of 8 wt % yohimbine, 40 wt % propanediol, and 37 wt % glycerol, and the balance is a flavoring agent.

Embodiment 3: The aerosol-forming substrate is composed of 6 wt % yohimbine, 40 wt % propanediol, and 37 wt % glycerol, and the balance is a flavoring agent.

Embodiment 4: The aerosol-forming substrate is composed of 4 wt % yohimbine, 40 wt % propanediol, and 37 wt % glycerol, and the balance is a flavoring agent.

Embodiment 5: The aerosol-forming substrate is composed of 3 wt % yohimbine, 40 wt % propanediol, and 37 wt % glycerol, and the balance is a flavoring agent.

Embodiment 6: The aerosol-forming substrate is composed of 1.5 wt % yohimbine, 40 wt % propanediol, and 37 wt % glycerol, and the balance is a flavoring agent.

Embodiment 7: The aerosol-forming substrate is composed of 0.5 wt % yohimbine, 40 wt % propanediol, and 37 wt % glycerol, and the balance is a flavoring agent.

Embodiment 8: The aerosol-forming substrate is composed of 0.1 wt % yohimbine, 40 wt % propanediol, and 37 wt % glycerol, and the balance is a flavoring agent.

Embodiment 9: 3 wt % yohimbine is selected as the aerosol-forming substrate, and a mixture of propanediol and glycerol with a mass ratio of about 7:3 is selected as the solvent.

Embodiment 10: 3 wt % yohimbine is selected as the aerosol-forming substrate, and a mixture of propanediol and glycerol with a mass ratio of about 3:7 is selected as the solvent.

Embodiment 11: 3 wt % pharmaceutically acceptable benzoate of yohimbine is selected as the aerosol-forming substrate, and a mixture of glycerol and propanediol with a mass ratio of about 1:1 is selected as the solvent. The pharmaceutically acceptable benzoate of yohimbine may be manufactured by mixing yohimbine alkaloid with benzoic acid.

Embodiment 12: 3 wt % pharmaceutically acceptable hydrochloride of yohimbine is selected as the aerosol-forming substrate, and a mixture of glycerol and propanediol with a mass ratio of about 1:1 is selected as the solvent. The pharmaceutically acceptable hydrochloride of yohimbine may be manufactured by separation and purification from the bark of the Rubiaceae Corunant Yohimbine tree, or may be obtained through the existing chemical synthesis method, and may further be purchased from the market.

Embodiment 1 to Embodiment 12 are inhaled by using a smoking machine according to international standards, and the aerosol vaporized by the aerosol-forming substrate is collected by using a Cambridge filter, then extracted by using an organic solvent, filtered, and analyzed and calculated through HPLC.

The conversion rate is calculated as follows. A weight difference m_0 of the Cambridge (that is, the weight of the collected aerosol) is calculated. An aerosol-forming substrate m₁ with the same weight is obtained for extraction by using an organic solvent with the same volume, and then an extract obtained is subjected to HPLC. A ratio of a chromatographic peak area A₁ to a concentration C₁ of the aerosol-forming substrate in the HPLC is multiplied by a chromatographic peak area A₀ of the aerosol, and then the product is divided by the concentration C₀ of the aerosol, to obtain the conversion rate. The specific calculation formula is as follows:

$$w \% = \frac{{C}_{1} \times {A}_{0}}{{A}_{1} \times {C}_{0}} \times 100 \% ,$$

where C₀=m₀/v, and C₁=m₁/v.

The calculation of the pharmaceutically acceptable salt of yohimbine is similar to above.

Different electronic vaporization devices are used for inhalation. An electronic vaporization device 1 adopts a vaporizer with a cotton core structure, an electronic vaporization device 2 adopts a vaporizer with a ceramic structure, and an electronic vaporization device 3 adopts a vaporizer with a cotton-covered ceramic structure. The vaporizer with the above structure may be learned from the prior art, and the details are not described herein.

For international standards involved, reference may be made to:

CORESTA RECOMMENDED METHOD No 81, Afnor standardization XP D90-300-3, International Standard ISO 20768:2018, and PD CEN/TR 17236:2018.

| | |
|---|---|
| Puff duration | 3.0 s ± 0.1 s |
| Puff volume | 55 mL ± 0.3 mL |
| Puff frequency | 30 s ± 0.5 s |
| Number of puffs of each group | 20 |
| Group interval time | 300 s ± 120 s |
| Maximum flow | 18.5 mL/s ± 0.1 mL/s |
| Pressure drop | <50 hPa |
| Group | 5 |
| Total number of puffs | 100 |
| Total duration of vaporization | 300 s |

Experimental test results: Different electronic vaporization devices are used for puffing, and corresponding conversion rates (k1-k3) thereof are calculated respectively. An average of the conversion rates is calculated based on the conversion rates corresponding to different electronic vaporization devices. The results are shown in the following table:

| | Conversion rate k1 | Conversion rate k2 | Conversion rate k3 | |
|---|---|---|---|---|
| | by using the | by using the | by using the | |
| | electronic | electronic | electronic | |
| | vaporization | vaporization | vaporization | |
| Embodiment | device 1 | device 2 | device 3 | Average |
| 1 | 31% | 40% | 30% | 34% |
| 2 | 37% | 45% | 36% | 39% |
| 3 | 41% | 50% | 40% | 44% |
| 4 | 45% | 52% | 43% | 47% |
| 5 | 48% | 55% | 49% | 51% |
| 6 | 56% | 68% | 58% | 61% |
| 7 | 65% | 72% | 66% | 68% |
| 8 | 68% | 75% | 72% | 72% |
| 9 | 67% | 78% | 55% | 67% |
| 10 | 45% | 55% | 48% | 49% |
| 11 | 62% | 78% | 59% | 66% |
| 12 | 46% | 65% | 60% | 57% |

It can be seen from the above experimental test results:
1) In Embodiment 1 to Embodiment 8, with the increase of the content of yohimbine in the aerosol-forming substrate, the conversion rate (average value) tends to decline. When the content of yohimbine in the aerosol-forming substrate reaches 10%, the conversion rate (average value) is about 34%.
2) In Embodiment 5 and Embodiment 9 to Embodiment 10, the content of propanediol in the solvent has great influence on the conversion rate. When the mass ratio of propanediol to glycerol is 7:3, the conversion rate (average value) can reach 67%, which is higher than the conversion rate in Embodiment 5 or Embodiment 10.
3) In Embodiment 5 and Embodiment 11 to Embodiment 12, the conversion rate (average value) of the pharmaceutically acceptable salt of yohimbine is generally high, and the conversion rate in Embodiment 11 or Example 12 is higher than the conversion rate in Embodiment 5. Yohimbine is a pharmaceutically acceptable benzoate, and the conversion rate (average value) may reach 66%.

### Implementation II:

Implementation II of this application provides an aerosol generation system, including an electronic vaporization device and the aerosol-forming substrate described in Implementation I.

The electronic vaporization device is configured to heat and vaporize the aerosol-forming substrate to generate an aerosol.

This written description uses examples to disclose this application, including the best mode, and also to enable any person skilled in the art to manufacture and use this application. The patentable scope of this application is defined by the claims, and may include another example that occur to a person skilled in the art. Such other examples are intended to be within the scope of the claims if they have structural elements that do not differ from the literal language of the claims, or if they include equivalent structural elements with insubstantial differences from the literal language of the claims. To the extent that no inconsistency arises, all citations referenced herein are incorporated herein by reference.

1. An aerosol-forming substrate, comprising yohimbine or a pharmaceutically acceptable salt of yohimbine or yohimbine hydrate, wherein
based on a total mass of the aerosol-forming substrate, a mass percent of the yohimbine or the pharmaceutically acceptable salt of the yohimbine or the yohimbine hydrate is in a range of 0.1%-10%.

2. The aerosol-forming substrate according to claim 1, wherein the pharmaceutically acceptable salt is selected from at least one of the following:
hydrochloride, hydrobromide, benzoate, hydrofluoride, sulfate, nitrate, phosphate, formate, acetate, propionate, oxalate, malonate, succinate, fumarate, maleate, lactate, malate, tartrate, citrate, picrate, mesylate, esilate, benzene sulfonate, aspartate, and glutamate.

3. The aerosol-forming substrate according to claim 2, wherein the pharmaceutically acceptable salt comprises benzoate.

4. The aerosol-forming substrate according to claim 1, further comprising a solvent, wherein the solvent is selected from at least one of the following:
propanediol, butanediol, pentanediol, hexanediol, 1,2,4-butanetriol, dipropylene glycol, diethylene glycol, triethylene glycol, polyethylene glycol 200, polyethylene glycol 400, glycerol triacetate, glycerol, dipropylene glycol ether, ethanol, water, triethyl citrate, caprylic capric triglyceride, isopropanol, sweet orange oil, lemon oil, peppermint oil, palm oil, peanut oil, corn oil, salad oil, xylitol, sorbitol, and erythritol.

5. The aerosol-forming substrate according to claim 4, wherein the solvent comprises propanediol, and a mass percent of the propanediol in the aerosol-forming substrate is in a range of 20%-60%, preferably 20%-50%, further preferably 20%-45%, Page 3 further preferably 25%-45%, further preferably 30%-45%, and further preferably 35%-45%.

6. The aerosol-forming substrate according to claim 5, wherein the solvent comprises glycerol, and a mass percent of the glycerol in the aerosol-forming substrate is in a range of 10-60%.

7. The aerosol-forming substrate according to claim 6, wherein a mass ratio of the propanediol to the glycerol is greater than 1, preferably 1.1-4:1, and further preferably 2-4:1.

8. The aerosol-forming substrate according to claim 1, further comprising a flavoring agent.

9. The aerosol-forming substrate according to claim 1, wherein the aerosol-forming substrate does not comprise nicotine and/or nicotine salts.

10. An aerosol generation system, comprising an electronic vaporization device and the aerosol-forming substrate according to claim 1, wherein
the electronic vaporization device is configured to vaporize the aerosol-forming substrate to generate an aerosol.

11. The aerosol-forming substrate according to claim 1, wherein
based on a total mass of the aerosol-forming substrate, a mass percent of the yohimbine or the pharmaceutically acceptable salt of the yohimbine or the yohimbine hydrate is in a range of 0.1%-8%.

12. The aerosol-forming substrate according to claim 1, wherein
based on a total mass of the aerosol-forming substrate, a mass percent of the yohimbine or the pharmaceutically acceptable salt of the yohimbine or the yohimbine hydrate is in a range of 0.1%-6%.

13. The aerosol-forming substrate according to claim 1, wherein
based on a total mass of the aerosol-forming substrate, a mass percent of the Page 4 yohimbine or the pharmaceutically acceptable salt of the yohimbine or the yohimbine hydrate is in a range of 0.1%-5%.

14. The aerosol-forming substrate according to claim 1, wherein
based on a total mass of the aerosol-forming substrate, a mass percent of the yohimbine or the pharmaceutically acceptable salt of the yohimbine or the yohimbine hydrate is in a range of 0.5%-5%.

15. The aerosol-forming substrate according to claim 1, wherein
based on a total mass of the aerosol-forming substrate, a mass percent of the yohimbine or the pharmaceutically acceptable salt of the yohimbine or the yohimbine hydrate is in a range of 1%-5%.

16. The aerosol-forming substrate according to claim 1, wherein
based on a total mass of the aerosol-forming substrate, a mass percent of the yohimbine or the pharmaceutically acceptable salt of the yohimbine or the yohimbine hydrate is in a range of 2%-5%.

17. The aerosol-forming substrate according to claim 5, wherein the solvent comprises glycerol, and a mass percent of the glycerol in the aerosol-forming substrate is in a range of 10%-50%.

18. The aerosol-forming substrate according to claim 5, wherein the solvent comprises glycerol, and a mass percent of the glycerol in the aerosol-forming substrate is in a range of 10%-40%.

19. The aerosol-forming substrate according to claim 5, wherein the solvent comprises glycerol, and a mass percent of the glycerol in the aerosol-forming substrate is in a range of 20%-40%.

20. The aerosol-forming substrate according to claim 5, wherein the solvent comprises glycerol, and a mass percent of the glycerol in the aerosol-forming substrate is in a range of 30%-40%.
